(19) United States
(12) Patent Application Publication
(10) Pub. No.: US 2019/0023679 A1
(43) Pub. Date: Jan. 24, 2019

(54) METHOD FOR SYNTHESISING AMBROXIDE FROM AGERATINA JOCOTEPECANA

(71) Applicant: UNIVERSIDAD MICHOACANA DE SAN NICOLÁS DE HIDALGOú, Morelia, Michoacán (MX)

(72) Inventors: Rosa Elva Norma DEL RÍO TORRES, Morelia, Michoacán (MX); Mario Armando GÓMEZ HURTADO, Morelia, Michoacán (MX); Ana Ixchel PÉREZ GUTIÉRREZ, Morelia, Michoacán (MX); Janett Betzabe GONZÁLEZ CAMPOS, Morelia, Michoacán (MX)

(21) Appl. No.: 15/769,785

(22) PCT Filed: Nov. 18, 2015
(86) PCT No.: PCT/IB2015/058939

§ 371 Date: Sep. 28, 2018

(30) Foreign Application Priority Data
Oct. 23, 2015 (MX) MX/A/2015/014945

(51) Int. Cl. C07D 307/92; B01J 20/10; B01J 20/291

(52) U.S. Cl. CPC C07D 307/92; B01J 20/103; B01J 2220/58; B01J 2220/54; B01J 20/291

(57) ABSTRACT

The present invention is related to a process for obtaining the (−)-13,14,15,16-tetranor-8a,12-labdanediol compound from the Ageratina jocotepecana plant, the process comprises the steps of a) obtaining an organic concentrated extract from the Ageratina jocotepecana shoot system; b) subjecting the organic concentrated extract to column chromatography in order to elute a fraction with the (−)-13,14,15,16-tetranor-8a,12-labdanediol compound; c) separating the eluted fractions comprising the (−)-13,14,15,16-tetranor-8a,12-labdanediol compound; and d) evaporating the organic solvent to yield the (−)-13,14,15,16-tetranor-8a,12-labdanediol compound in a solid form.

### FIELD OF THE INVENTION

The present invention is related to techniques for the synthesis of chemical compounds, and more particularly it is related to a synthesis process for (−)-ambrox from Ageratina jocotepecana.

### BACKGROUND OF THE INVENTION

(−)-Ambrox corresponds to the (−)-3a,6,6,9a-tetramethyldodecahydronaphto-(2,1-b)furane compound, having the chemical structure:

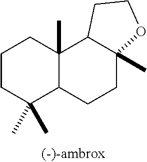

(−)-Ambrox is a chemical compound mainly employed as a fixative in the perfume industry, which was traditionally obtained from the sperm whale (Physeter macrocephalus) gastrointestinal tract, an endangered species. As a consequence, multiple alternative procedures have been developed for the synthesis and obtainment of this compound using other natural products as starting materials, mainly terpenes, which can be obtained from plant-species extracts. However, as will be detailed later, these procedures involve several reaction steps, which range from three to twenty-five. Additionally, the respective routine isolation and purification processes should be considered, both for the synthesis intermediates and the final product.

It is possible to obtain ambrox in a completely synthetic way from monocyclohomofarnesyl acid or derivatives thereof such as monocyclohomofarnesol. This way, Ambrox DL® of Firmenich (>50% (±)-ambrox and <50% diastereomers) and Cetalox® products are synthesized. There is also the commercial product Cetalox Laevo® (>99% (−)-ambrox) presumably produced via optic resolution of the intermediate (±)-sclareolide. These synthetic products are obtained from processes which do not allow to obtain the enantiomerically pure (−)-ambrox, thus being commercialized in racemic form, or optionally, by applying costly and complex chiral-resolution methods.

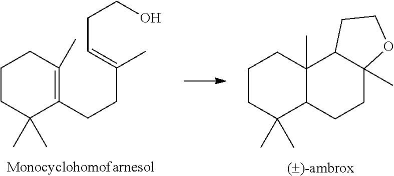 [SMILES: CC1=C(CC/C(C)=C/CCO)C(C)(C)CCC1 ; CC1(C)CCCC2(C)C1CCC1(C)OCCC12]

Another procedure for obtaining ambrox is disclosed in Spanish Patent ES2069469A1, which consists in a five-step semi-synthesis from naturally occuring trans- and cis-communic diterpenic acids, obtained from the species of genus Juniperus, for preparing (−)-ambrox. In the first step, degradation of the communic acid double bond Δ¹² is achieved in a considerably regioselective manner by reductive ozonolysis at low temperture, to obtain the homosesquiterpenic alcohol, which is treated with acid in the presence of nitromethane, in order to quantitatively and entirely-stereoselectively cycle it yielding the epoxide, and finally, deprotecting the methyl groups to obtain the (−)-ambrox compound. Said procedure is complex, time consuming and expensive, since it requires several reaction steps and reagents to obtain the (−)-ambrox compound.

A further synthesis was reported by Rosana A. Giacomini et. al., “Synthesis of ambergris odorant ent-ambrox”, ARKIVOC 2003 (x), 314-322, from ozoic acid, which es subjected to a diazomethane esterification to obtain a new diterpene, and then the ozoic acid methyl ester is subjected to a series of complex reactions to result in ent-ambrox, among the reactions there is the use of an ozone stream followed by treatment with PPh3, then treatment with ethylene glycol in benzene and camphorsulfonic acid, and the Wittig reaction using trimethylphosphonium bromide and n-BuLi as base. Said process is also complex, time consuming and expensive.

In yet another ambrox synthesis procedure disclosed in the U.S. Pat. No. 5,616,737, the cyclization of decahydro-2-hydroxi-2,5,5,8-tetramethyl-1-naphthalene ethanol compound is carried out by heating it to its melted state between 80° C. to 200° C., in the presence of 10 to 100% by weight, diol-based, of an active-acidic aluminum oxide commercially supplied for column chromatography, which has as disadvantage the high cost of reagents and equipment necessary to carry out the process, as well as the use of a very specific alumina catalyst, which is pre-treated with chlorhydric acid.

Moreover, there are various synthesis methods using sclareol as raw material for the synthesis of (−)-ambrox, which have the disadvantage that the sclareol is an expensive material, present only in very small amounts in the Salvia sclarea essential oil. In addition, the sclareol compound is not able to be converted into ambrox by a single chemical reaction, instead it requires at least one intermediate such as typically methyl-ketones, 13,14,15,16-tetranor-8a,12-labdanediol or norambreinolide.

For example, according to U.S. Pat. No. 5,463,089, the (−)-sclareol compound is subjected to oxidation or catalytic rearrangement with OsO₄ to form methyl-ketone intermediates, which are converted by the Baeyer-Villiger oxidation in order to produce acetate intermediates, which are converted to the desired (−)-ambrox compound.

As another example, in patent US20100248316 it is disclosed that (−)-sclareol may be treated with the Hyphozyma roseoniger microorganism to obtain the 13,14,15,16-tetranor-8a,12-labdanediol compound, which is then cyclized in the presence of a zeolite to yield the (−)-ambrox compound. This process requires very long reaction times and the zeolite activation increases the synthesis costs.

Alternatively, as described in Spanish Patent Application ES2044780A1, the (−)-sclareol compound can be subjected to a degradation process of the C₁₂-C₁₃ bond, by treatment with osmium tetroxide-sodium peryodate at 45° C., and the reduction with lithium aluminum hydride to provide the 13,14,15,16-tetranor-8a,12-labdanediol compound, which is then cyclized in the presence of p-toluenesulfonyl chloride and pyridine, yielding the (−)-ambrox compound. In another procedure described in the same application, 13,14,15,16-tetranor-8a,12-labdanediol is obtained from the cis-abienol compound, which is subjected to the side chain degradation by reducting ozonolysis at low temperature, to form the 13,14,15,16-tetranor-8a,12-labdanediol compound, which is transformed into (−)-ambrox as described above.

Another known typical process for the synthesis of (−)-ambrox comprises oxidizing the (−)-sclareol compound with chromic acid to synthesize (+)-norambreinolide, then reducing the (+)-norambreinolide with lithium aluminum hydride MAIN or sodium boranate or (NaBH₄), and cycling the reduction product with an acid or with bromotrichloromethane and triphenylphosphine in methylene chloride at reflux to obtain (−)-ambrox [Dragoco Report, 11/12, 276-283 (1979) and G. Ohloff, Fortschr. Chem. Forsch. 12/2, (1969)]. However, the use of chromic acid as reagent is dangerous, and its disposal is problematic. In addition, they use other reagents such as lithium aluminum hydride, which es highly inflammable and non-suitable for industrial use. Then, the (−)-ambrox produced by these methods is very expensive.

As described in Spanish Patent ES2195777, norambreinolide is also obtained from sclareol by reacting it with potassium permanganate at room temperature. Norambreinolide is treated with sodium borohydride in the presence of zinc iodide, or with potassium borohydride in the presence of boron trifluoride to obtain the (−)-ambrox compound; or with vitride o potassium borohydride to obtain 13,14,15,16-tetranor-8a,12-labdanediol, which is reacted with bromotrichloromethane and triphenylphosphine in methylene chloride at reflux to yield (−)-ambrox. These methods have the drawback of using numerous reagents in each reaction step, as well as some toxic reagents such as triphenylphosphine.

Regarding norambreinolide, the documents Chemistry Letters, pp. 757-760 (1981) and pp. 729-732 (1983), propose a synthesis method of (t)-norambreinolide, which comprises reacting (±)-trans-β-monocycle-homofarnesylic acid in dichloromethane in the presence of tin tetrachloride.

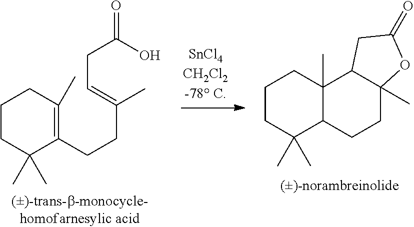 [SMILES: CC1=C(CC/C(C)=C/CC(=O)O)C(C)(C)CCC1 ; CC1(C)CCCC2(C)C1CCC1(C)OC(=O)CC12]

However, this method requires an extremely low temperature of −78° C., which is non-suitable for industrial practice. Additionally, isomers of (±)-norambreinolide are formed, and the yielding of the (±)-norambreinolide compound and its isomers is as low as 57%.

On the other hand, there are various procedures to carry out the cyclization of the 13,14,15,16-tetranor-8a,12-labdanediol compound to obtain the (−)-ambrox compound, some of them were already mentioned above, and others are described below.

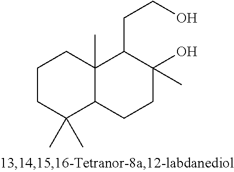 [SMILES: CC1(C)CCCC2(C)C1CCC(C)(O)C2CCO]

Patente EP204009 describes the cyclization of 13,14,15,16-tetranor-8a,12-labdanediol with arylsulfonyl chloride in the presence of an acidic compound, such as HCl and acid ion-exchangers, or in the presence of bases, such as pyridine and NaOH. However, said procedures are very time consuming, expensive, complex, and a suitable purity of the resulting compound is not obtained.

On the other hand, European Patent application EP0165458A2 discloses that cyclization of (±)-13,14,15,16-tetranor-8a,12-labdanediol is carried out in the presence of a base and p-toluenesulfonyl chloride, wherein the racemate of (±)-13,14,15,16-tetranor-8a,12-labdanediol can be synthesized from compounds known as (±)-trans-β-monocyclo-homofarnesylic acid or its esters and the (±)-trans-β-monocyclo-homofarnesylic acid can in turn be synthesized from dihydro-β-ionone, which is non-expensive and easy to synthesize or obtain. However, the process requires several steps and reagents, which increase the process costs. Additionally, pyridine is used as a base in one step of the process, which has an unpleasant odor, so the quality of the resulting essence has to be adjusted and, in an aqueous treatment, the pyridine recycling is very difficult due to its solubility in water, which leads to an increased cost.

Cyclization of 13,14,15,16-tetranor-8a,12-labdanediol can also be carried out in the presence of p-toluenesulfonic acid or sulfuric acid, which disadvantageously results in the dehydration of the tertiary hydroxy group, thereby causing selectivity loss and low yieldings [V. E. Sibiertseva et. al., Maslo-Zhir, Prom. st., 1979 (12), 25-26, Russian Patents SU345183 (from 1968), SU910561 (from 1980) and SU529166 (from 1975)] and Spanish Patent ES432815.

Also, the 13,14,15,16-tetranor-8a,12-labdanediol may be cycled with p-toluene-sulfonyl chloride in pyridine (R. C. Cambie et. al., Aust. J. Chem. 24 (1971), pp. 583-591 and 2365-2377), or with POCl₃ in anhydride pyridine (German Patent DE-A 3240054). Such procedures have the disadvantage of using pyridine, as well as long reaction times and low yielding.

According to Russian Patent SU988817, cyclization can also be carried out with trimethylchlorosilane in dimethylsulfoxide (DMSO), but the DMSO has to be recycled thoroughly after water treatment due to its solubility in water, causing issues with waste-water, and further it is necessary to purify the obtained raw-essence after the reaction in a quality being suitable in both chemical and olfactory aspects. In addition, the trimethylchlorosilane is industrially difficult to handle, since it is very corrosive and toxic.

Also, for the cyclization of 13,14,15,16-tetranor-8a,12-labdanediol, sulfuric acid, phosphoric acid or polyphosphoric acid-loaded white-soil, alumina or silicon from 1 to 20% by weight can be used as catalysts, however, the use of said acids is disadvantageous since the handling of their residues is difficult at a commercial scale (Chem. Abstr. 105 (1986) 134193k).

Another example of cyclization procedure of 13,14,15,16-tetranor-8a,12-labdanediol is disclosed in German Patent DE3912318, which is carried out with particles having 60-80% by weight Al₂O₃ and 0.40 a 0.6% by weight chlorhydric acid, which has the inconvenience of being not selective enough for the olfactory requirements, and a very specific alumina catalyst pre-treated with chlorhydric acid has to be used, which increases its costs.

Regardless of the above, the Ageratina jocotepecana plant has recently been studied as it comprises significant amounts of terpenes that may be useful in different industry branches. The Ageratina jocotepecana plant is a Mexican endemic plant being found in the road between Morelia and Zacapu.

According to document “Absolute Configuration of (13R)- and (13S)-Labdane Diterpenes Coexisting in Ageratina jocotepecana”, Journal of Natural Products, 2014, 77, pp. 1005-1012, the Ageratina jocotepecana plant contains several labdane diterpenes with antibacterial activity corresponding to normal-(5S,10S) series, as well as C-13 epimers. However, in this paper it is not mentioned that the diterpenoid compounds may be used for preparing (−)-ambrox.

The paper published by Ana I. Pérez-Gutiérrez, Alejandra León Hernández, Juan D. Hernández-Hernández, Luisa U. Román-Marin, Carlos M. Cerda-Garciá-Rojas, Pedro Joseph-Nathan, Rosa E. del Rio, entitled “Ageratina jojotepecana una nueva fuente para la obtención de ent-ambrox”, Biol. Soc. Quím. Méx. 2010, 4 (Special Number), suggests the isolation of an hexanic-extract tetranorlabdanediol from Ageratina jocotepecana stems, which turned out to be a new source for the synthesis of ambrox derivatives. However, said document does not describe the tetranorlabdanediol structure, neither it describes the conditions for the obtainment of said tetranorlabdandiol and ambrox derivatives.

The paper published by Sergio I. Martinez-Guido, J. Betzabe González-Campos, Rosa E. del Río, José M. Ponce-Ortega, Fabricio Nápoles-Rivera, Medardo Serna-González, and Mahmoud M. El-Halwagi. “A Multiobjective Optimization Approach for the Development of a Sustainable Supply Chain of New Fixative in the Perfume Industry”, ACS Sustainable Chemistry Engineering, Sep. 2, 2014, pp. 2380-2390, mentions that Ageratina jocotepecana extracts having (−)-13,14,15,16-tetranor-8a,12-labdanediol, from which (−)-ambrox is obtained by a cyclization reaction with p-toluenesulfonic acid in benzene. However, said document does not describe nor suggests the conditions for obtaining (−)-13,14,15,16-tetranor-8a,12-labdanediol from Ageratina jocotepecana.

Although there are several reported synthesis for obtaining (−)-ambrox, sclareol is still the most widely used compound as starting material for the synthesis of (−)-ambrox. However, the reported synthesis methods from this compound show as a main disadvantage a high reagent consumption, which results in high costs, plus the toxicity risk for the people in charge of the procedure, and then adding the time invested to obtain the product, since these synthesis usually involve a wide range of reactions in their development.

On the other hand, the other known processes that are not sclareol-based comprise expensive and time consuming procedures, or which may generate a considerable amount of undesired pollutants and/or by-products.

According to the above-mentioned, there is still a need of a process allowing to obtain the (−)-ambrox compound in a simpler, economic, sustained manner and without endangering the sperm whale for its obtainment. Moreover, there is still the need to obtain the 13,14,15,16-tetranor-8a,12-labdanediol compound in a environmental friendly and economic manner for its use in the synthesis of (−)-ambrox.

### OBJECTS OF THE INVENTION

Taking into account the drawbacks of the prior art, it is an object of the present invention to provide an efficient process for obtaining (−)-ambrox from the Ageratina jocotepecana plant, which involves the minimum possible reaction steps in order to reduce costs, times, and environmental pollution.

It is another object of the present invention to provide a process for obtaining an organic concentrated extract from the Ageratina jocotepecana plant which contains the (−)-13,14,15,16-tetranor-8a,12-labdanediol compound, which is suitable for the synthesis of the (−)-ambrox compound.

It is yet a further object of the present invention to provide a purification method for the (−)-13,14,15,16-tetranor-8a,12-labdanediol compound.

It is a further object of the present invention to provide the (−)-13,14,15,16-tetranor-8a,12-labdanediol compound, which is obtained from the organic concentrated extract of Ageratina jocotepecana, for its use in a process for obtaining (−)-ambrox.

It is yet a further object of the present invention to avoid using the sperm whale (Physeter macrocephalus) as a source for the obtainment of (−)-ambrox, thereby contributing to its protection.

### SUMMARY OF THE INVENTION

The present invention comprises a process for obtaining the (−)-13,14,15,16-tetranor-8a,12-labdanediol compound from the Ageratina jocotepecana plant, which is a valuable compound for preparing the (−)-ambrox compound, wherein the process comprises the following steps: a) obtaining an organic concentrated extract from the shoot system of the Ageratina jocotepecana plant. B. L. Turner; b) subjecting the organic concentrated extract to silica gel column chromatography with hexane, ethyl acetate and hexane:ethyl acetate mixtures as mobile phase, including a mixture from 60:40 a 40:60 hexane:ethyl acetate to elute a fraction with the (−)-13,14,15,16-tetranor-8a,12-labdanediol compound; c) separating the eluted fractions containing the (−)-13,14,15,16-tetranor-8a,12-labdanediol compound; and d) evaporating the hexane:ethyl acetate mixture in the separated fractions to yield the (−)-13,14,15,16-tetranor-8a,12-labdanediol compound.

Another aspect of the present invention comprises a process for obtaining the (−)-ambrox compound comprising the steps of: a) obtaining the (−)-13,14,15,16-tetranor-8a,12-labdanediol compound from the Ageratina jocotepecana plant B. L. Turner in accordance with the above-described principles of the present invention; and b) subjecting the (−)-13,14,15,16-tetranor-8a,12-labdanediol compound to a cyclization step to obtain the (−)-ambrox compound.

### DETAILED DESCRIPTION OF THE INVENTION

The present invention is related to a process for obtaining the (−)-13,14,15,16-tetranor-8a,12-labdanediol compound from the Ageratina jocotepecana plant, which is a valuable compound in the preparation of the (−)-ambrox compound, wherein the process comprises the steps of: a) obtaining an organic concentrated extract of the Ageratina jocotepecana shoot system; b) subjecting the organic concentrated extract to column chromatography for eluting a fraction with the (−)-13,14,15,16-tetranor-8a,12-labdanediol compound; c) separating the eluted fractions comprising the (−)-13,14,15,16-tetranor-8a,12-labdanediol compound; and d) evaporating the organic solvent to yield the (−)-13,14,15,16-tetranor-8a,12-labdanediol compound.

In a preferred embodiment, the organic concentrated extract from the Ageratina jocotepecana shoot system is obtained by a process comprising the following steps: a) drying the Ageratina jocotepecana plant shoot system, wherein the Ageratina jocotepecana shoot system comprises the aerial parts of the plant, such as flowers, leaves and/or stems; b) macerating or refluxing the Ageratina jocotepecana plant shoot system in an organic solvent for a period of time from 6 to 72 hours; c) filtering out the macerated or refluxed extract of step (b) to obtain an organic extract of Ageratina jocotepecana; and d) evaporating the organic solvent from the Ageratina jocotepecana organic extract to obtain an organic concentrated extract from the Ageratina jocotepecana shoot system.

In a preferred embodiment, the organic solvent used for the obtainment of the organic concentrated extract from Ageratina jocotepecana shoot system of step (a) is selected from the group consisting of hexane, dichloromethane, ethyl acetate, chloroform, methanol and/or mixtures thereof.

In a preferred embodiment, the step of column chromatography is carried out in silica gel as stationary phase. In a more preferred embodiment, the chromatography step uses a mobile phase comprising hexane, ethyl acetate and mixtures thereof in different ratios and in a polarity increasing order.

In a preferred embodiment, the eluted fractions comprising mixtures of hexane:ethyl acetate in a ratio from 60:40 to 40:60 (i.e., 3:2 a 2:3) hexane:ethyl acetate are separated, to elute a fraction with the (−)-13,14,15,16-tetranor-8a,12-labdanediol compound, preferably in a ratio of 50:50 hexane:ethyl acetate (i.e., 1:1).

In a still more preferred embodiment, the evaporation step of the organic solvent to obtain the (−)-13,14,15,16-tetranor-8a,12-labdanediol compound is carried out at a temperature from 35 to 45° C., more preferably at 40° C., and reduced pressure.

According to the principles of the present invention, the (−)-13,14,15,16-tetranor-8a,12-labdanediol compound is obtained in solid or semi-solid form, wherein the solid form can comprise the crystalline or amorphous form, and wherein the semi-solid form can be a syrup, thick suspension or paste.

In another aspect of the present invention, the step of subjecting the (−)-13,14,15,16-tetranor-8a,12-labdanediol compound to a cyclization step is added to the above-disclosed process in order to obtain a raw product of (−)-ambrox compound.

The cyclization step comprises well known methods to carry out the cyclization of the (−)-13,14,15,16-tetranor-8a,12-labdanediol compound in order to obtain a raw product comprising the (−)-ambrox compound, said methods comprise dissolving the (−)-13,14,15,16-tetranor-8a,12-labdanediol compound in a suitable organic solvent, then adding acid and/or base and/or a suitable reagent to carry out the cyclization. Preferably, the organic solvent is selected from the group consisting of hexane, benzene, dimethylsulfoxide, ethyl acetate, or mixtures thereof. The acid is preferably selected from a Lewis acid or p-toluensulfonic acid. The base is preferably selected from pyridine or NaOH. Finally, the suitable reagent to carry out the cyclization is selected from POCl₃, trimethylchlorosilane, tosyl chloride, among other compounds able to cycling a diol, and more preferably a labdanediol.

In a preferred embodiment of the process of the present invention, the (−)-ambrox compound raw product is subjected to a purification procedure to obtain the (−)-ambrox compound in a crystalline form, for which various different methods known for purifying organic chemical compounds may be used. Preferably, purification by column chromatography is preferred.

In a more preferred embodiment, the purification process comprises a silica gel column as stationary phase.

In a yet more preferred embodiment, the purification process uses a mobile phase comprising hexane, ethyl acetate and mixtures thereof in different ratios and in a polarity increasing order.

In a still more preferred embodiment, the fraction comprising the polarity is separated from a mixture of 4:1 hexane:ethyl acetate.

In a still more preferred embodiment, the evaporation step of the organic solvent to obtain (−)-ambrox crystals is carried out at a temperature from 35 to 45° C., more preferably at 40° C., and reduced pressure.

The present invention will be better understood from the following examples, which are given for illustrative purposes only in order to allow a better understanding of the preferred embodiments of the present invention, without implying that there are no other non-illustrated embodiments which can be taken into practice based on the above detailed description.

### EXAMPLES

### Example 1

For the obtainment of the organic concentrated extract from the Ageratina jocotepecana plant shoot system, the shoot system comprises the aerial parts of the plant, such as: flowers, leaves and/or stems previously separated and shadow-dried, during 240 hours at a temperature from 10° C. to 28° C., 320 g of Ageratina jocotepecana plant shoot system are weighed (amount of plant), 1500 mL of dichloromethane are added, the mixture is left in maceration at 25° C. during 72 hours, after this time, the macerated extract is filtered out to obtain the organic extract, which is concentrated in a rotary evaporator at 40° C. and reduced pressure.

### Example 2

For the obtainment of the organic concentrated extract from the Ageratina jocotepecana plant shoot system, the shoot system comprises the aerial parts of the plant, such as: flowers, leaves and/or stems previously separated and shadow-dried, during 240 hours at a temperature from 10° C. to 28° C., 450 g of Ageratina jocotepecana plant shoot system are weighed, 2000 mL of hexane are added, the mixture is subjected to reflux during 6, after this time, the mixture is filtered out to obtain the organic extract, which is concentrated in a rotary evaporator at 40° C. and reduced pressure.

### Example 3

The organic concentrated extract of Example 1 or Example 2 is subjected to a column chromatography process, which comprises a 70-230 mesh silica gel stationary phase (Aldrich®), and then adding the mobile phase comprising hexane-ethyl acetate gradient mixtures, with increasing polarity, in an amount sufficient to obtain 10 mL aliquots. The eluted fraction in the 50:50 ratio of hexane:ethyl acetate (i.e., 1:1) is separated, and said fraction is subjected to evaporation by means of a rotary evaporator at 40° C. and reduced pressure to remove the organic solvent to obtain the (−)-13,14,15,16-tetranor-8a,12-labdanediol compound in a colorless crystal form, having a melting point of 130-135° C.

### Example 4

320 g of room temperature, shadow-dried Ageratina jocotepecana stems are weighed, which are ground and subjected to extraction by maceration with 2 L hexane during three days, the macerated extract is filtered out and the organic solvent is evaporated in a rotatory evaporator at 40° C. and reduced pressure, thereby producing 20 g of organic concentrated extract.

2 g of organic concentrated extract are weighed and subjected to column chromatography using 15 g of 70-230 mesh silica gel as stationary phase (Aldrich®), a 1.5 cm diameter chromatography crystal column, and distilled hexane and ethyl acetate solvents and mixtures thereof as mobile phase, in different ratios, in an increasing polarity to obtain 10 mL fractions each; fractions having an hexane-ethyl acetate 1:1 polarity are extracted, the organic solvent is evaporated in a rotary evaporator at 40° C. and reduced pressure to yield the 13,14,15,16-tetranor-8a,12-labdanediol compound in a crystalline form in an amount of 100 mg.

### Example 5

0.1 g of the (−)-13,14,15,16-tetranor-8a,12-labdanediol compound (0.42 mmol) in crystalline form obtained in accordance with any one of Examples 3 or 4 are weighed, then it is dissolved in 20 mL of benzene, 40 mg of p-toluensulfonic acid (0.23 mmol) are added to carry out the cyclization reaction and to obtain 0.078 g of an (−)-ambrox compound raw product.

### Example 6

The raw product obtained according to Example 5 is purified by column chromatography to obtain the (−)-ambrox compound in a pure crystalline form, imputity-free, using a Merck® 230-400 mesh silica gel as stationary phase, and as mobile phase it comprises mixtures of hexane-ethyl acetate at different ratios in a polarity increasing order to obtain 10 mL fractions. Fractions comprising a 4:1 ratio of hexane-ethyl acetate are separated, and the solvent is evaporated by means of a rotatory evaporator at 40° C. and reduced pressure to form colorless crystals comprising the (−)-ambrox compound, showing a ¹H NMR (400 MHz, CDCl₃) with the following characteristic signals: δ ppm 3.92 (3H, m, H-12), 3.82 (1H, ddd, J=8 Hz, H-12″), 1.94 (1H, dt, J=11.5, 3.2, 3.2 Hz, H-7), 1.09 (3H, s, CH₃-17), 0.87 (3H, s, CH₃-18), 0.84 (3H, s, CH₃-19), 0.83 (3H, s, CH₃-20) and a specific rotation value of [α]_{D}=−8.6 (c=0.4, CHCl₃).

According to the above description, it may be seen that the process of the present invention has been developed to obtain the (−)-13,14,15,16-tetranor-8a,12-labdanodiol compound from the Ageratina jocotepecana plant, which is a suitable intermediate for the synthesis of the (−)-ambrox compound; and it will be apparent to one skilled in the art that the embodiments of the process for obtaining (−)-ambrox as described above, are only illustrative but non-limitative of the present invention, since several major changes in its details are possible without departing from the scope of the invention.

Therefore, the present invention should not be considered as restricted except for what the prior art demands and the scope of the appended claims.

1. A process for obtaining the (−)-13,14,15,16-tetranor-8a,12-labdanediol compound from the Ageratina jocotepecana plant, wherein the process comprises the steps of: a) obtaining an organic concentrated extract from the Ageratina jocotepecana shoot system; b) subjecting the organic concentrated extract to column chromatography to elute a fraction with the (−)-13,14,15,16-tetranor-8a,12-labdanediol compound; c) separating the eluted fractions comprising the (−)-13,14,15,16-tetranor-8a,12-labdanediol compound; and d) evaporating the organic solvent to yield the (−)-13,14,15,16-tetranor-8a,12-labdanediol compound in a solid form.

2. The process according to claim 1, wherein the organic concentrated extract from the Ageratina jocotepecana shoot system is obtained by a process comprising the following steps: a) drying the Ageratina jocotepecana plant shoot system, wherein the Ageratina jocotepecana shoot system comprises the aerial parts of the plant, such as flowers, leaves and/or stems; b) macerating or refluxing the Ageratina jocotepecana plant shoot system in an organic solvent for a period of time from 6 to 72 hours; c) filtering out the macerated or refluxed extract of step (b) to obtain an Ageratina jocotepecana organic extract; and d) evaporating the solvent from the Ageratina jocotepecana organic extract.

3. The process according to claim 1, wherein the organic solvent is selected from the group consisting of hexane, dichloromethane, ethyl acetate, chloroform, methanol and/or mixtures thereof.

4. The process according to claim 1, wherein the column chromatography step is carried out in silica gel as stationary phase.

5. The process according to claim 1, wherein the chromatography step uses a mobile phase comprising hexane, ethyl acetate and mixtures thereof in different ratios and in a polarity increasing order.

6. The process according to claim 1, wherein the eluted fractions comprising mixtures of hexane:ethyl acetate in a ratio from 60:40 to 40:60 (i.e., 3:2 a 2:3) of hexane:ethyl acetate, are separated.

7. The process according to claim 6, wherein preferably the eluted fractions comprising mixtures of hexane:ethyl acetate in a ratio of 50:50 of hexane:ethyl acetate (i.e., 1:1) are separated.

8. The process according to claim 1, wherein the evaporation step of the organic solvent to obtain the (−)-13,14,15,16-tetranor-8a,12-labdanediol compound is carried out at a temperature from 35 to 45° C., and reduced pressure.

9. The process according to claim 8, wherein the evaporation step of the organic solvent to obtain the (−)-13,14,15,16-tetranor-8a,12-labdanediol compound is carried out at a temperature of 40° C., and reduced pressure.

10. The process according to claim 1, wherein the (−)-13,14,15,16-tetranor-8a,12-labdanediol compound is obtained in a solid or semi-solid form, wherein the solid form comprises the crystalline or amorphous form, and wherein the semi-solid form is a syrup, a thick suspension or a paste.

11. A process for obtaining the (−)-ambrox compound from the Ageratina jocotepecana plant, wherein the process comprises the steps of: a) obtaining an organic concentrated extract from the Ageratina jocotepecana shoot system; b) subjecting the organic concentrated extract to column chromatography in order to elute a fraction with the (−)-13,14,15,16-tetranor-8a,12-labdanediol compound; c) separating the eluted fractions comprising the (−)-13,14,15,16-tetranor-8a,12-labdanediol compound; d) evaporating the organic solvent to obtain the (−)-13,14,15,16-tetranor-8a,12-labdanediol compound in a solid form; and e) subjecting the (−)-13,14,15,16-tetranor-8a,12-labdanediol compound to a cyclization step to yield the (−)-ambrox compound.

12. The process according to claim 11, wherein the cyclization step comprises a) dissolving the (−)-13,14,15,16-tetranor-8a,12-labdanediol compound in a suitable organic solvent; b) adding acid and/or base and/or a suitable reagent to carry out the cyclization.

13. The process according to claim 12, wherein the organic solvent is selected from the group consisting of hexane, benzene, dimethylsulfoxide, ethyl acetate, or mixtures thereof.

14. The process according to claim 12, wherein the acid is selected from the group consisting of a Lewis acid or p-toluensulfonic acid.

15. The process according to claim 12, wherein the base is selected from the group consisting of pyridine or NaOH.

16. The process according to claim 12, wherein the suitable reagent to carry out the cyclization is selected from the group consisting of POCl₃, trimethylchlorosilane, tosyl chloride or other compounds able to cycling a diol.

17. The process according to claim 11, wherein a purification step for the (−)-ambrox compound is optionally carried out.

18. The process according to claim 17, wherein the purification step of the (−)-ambrox compound is carried out by column chromatography.

19. The process according to claim 17, wherein the column chromatography comprises a silica gel column as stationary phase, and a mobile phase comprising hexane, ethyl acetate and mixtures thereof in different ratios, and in a polarity increasing order.

20. The process according to claim 19, wherein the fraction comprising the polarity of a 4:1 hexane:ethyl acetate mixture is separated.

21. The process according to claim 20, wherein the process comprises evaporating the mixture of hexane:ethyl acetate at a temperature from 35 to 45° C., and reduced pressure.

22. The process according to claim 21, wherein the evaporation is carried out at a temperature of 40° C., and reduced pressure.
